(19) United States
(12) Patent Application Publication
(10) Pub. No.: US 2015/0088317 A1
(43) Pub. Date: Mar. 26, 2015

(54) CONTACTLESS ELECTROCARDIOGRAPHIC SENSOR WITH MOISTURE GENERATOR

(71) Applicant: FORD GLOBAL TECHNOLOGIES, LLC, Dearborn, MI (US)

(72) Inventors: Benjamin EILEBRECHT, Herne NRW (DE); Lennart LEICHT, Aachen (DE); Marcel MATHISSEN, Wuerselen NRW (DE); Jeroen LEM, Maastricht (NL); Achim LINDNER, Euskirchen (DE); Rainer VOGT, Aachen NRW (DE); Steffen LEONHARDT, Aachen (DE); Marian WALTER, Aachen (DE)

(21) Appl. No.: 14/493,901

(22) Filed: Sep. 23, 2014

(30) Foreign Application Priority Data
Sep. 23, 2013 (DE) 102013219026.3

(51) Int. Cl. A61B 5/18; A61B 5/00; A61B 5/0408; G05D 22/02; B60H 3/02

(52) U.S. Cl. CPC A61B 5/18; G05D 22/02; B60H 3/02; A61B 5/0408; A61B 5/6893
USPC 700/276

(57) ABSTRACT

A sensor for a contactless electrocardiographic measurement of a person includes an electrode formed of a moisture-permeable material and having a measurement surface and an opposite surface. A moisture generator supplies moisture to the opposite surface, and a moisture sensor detects a moisture content of a microclimate at the measurement surface. A controller receives signals from the moisture sensor and activates the moisture generator based upon the signals to control the moisture content. The moisture generator may be a heating element heating a source of moisture; a pump activated pumping liquid from a reservoir to the electrode; an ultrasonic atomizer for atomizing liquid contained in a reservoir; an actuator varying an amount of a liquid-conducting material in contact with liquid contained in a reservoir; or a Peltier element operable to warm and thereby release moisture from a moisture-storing material adjacent to the electrode.

### CROSS-REFERENCE TO RELATED APPLICATIONS

This application claims foreign priority benefits under 35 U.S.C. §119(a)-(d) to DE 102013219026.3 filed Sep. 23, 2013, which is hereby incorporated by reference in its entirety

### TECHNICAL FIELD

The present invention relates to contactless electrocardiographic measurement of a person seated in a motor vehicle, and more specifically to a contactless electrocardiographic sensor having a moisture generator operative to increase the moistness of a microclimate adjacent to the measurement surface of the sensor.

### BACKGROUND

Measurement of the electrical potential, or electrical field strength, on the skin of a person by means of electrocardiographic sensors forms the basis of many medical diagnostic methods. In this way, for example, an electrocardiogram (ECG) may be recorded or the heart rate may be determined from the measured electrical potentials.

In conventional measurement methods for measuring the electrical potential on the skin, the latter is acquired by electrodes which are in direct electrical contact with the surface of the skin. An electrically conductive connection is thus established between the skin, on the one hand, and the electrode, on the other hand. In this case, however, it often proves difficult to ensure a sufficiently good electrical contact between the electrode and the skin, and therefore the body of the person being examined (the subject). Furthermore, the use of such diagnostic methods is also increasingly being provided in application fields in which direct access to the skin of the subject is not available, for example in vehicle applications for monitoring body functions and/or vital parameters of vehicle passengers on seats or bunks.

For example, U.S. Pat. No. 7,684,854 B2 discloses a sensor for contactless electrocardiographic measurement on a person. The person may in this case be on a stool, in a bed or on a vehicle seat. The electrocardiogram can be recorded from the body of the person wearing clothing without direct contact with the skin. The sensor comprises a flat electrically conductive electrode which comprises a measurement surface facing toward the person and a connection surface which faces away from the person, lies opposite the measurement surface and is electrically connected to a preamplifier. The electrode and the preamplifier of the sensor are enclosed by shielding.

Another contactless sensor for recording an electrocardiogram of a person is disclosed by EP 2 532 306 A1. The sensor comprises an electrically conductive electrode and a detection device, which is electrically connected to the electrode and is configured in order to amplify the signals received by the electrode. The sensor is intended to be arranged in a vehicle seat and to determine particular physiological parameters of a driver sitting on the vehicle seat.

DE 20 2012 001 096 U1 discloses capacitive sensors for capacitive recording of vital parameters of a driver of a vehicle. To this end, the sensors are fitted in or on the backrest of the seat of the vehicle. In particular, according to one embodiment it is proposed to arrange the sensors in or on the backrest of the seat while being distributed in two rows separated by a distance corresponding to the width of the spinal column of the driver. In each row, the sensors, with an area of from 16 to 36 cm², are arranged at equal distances of from 1 to 5 cm from one another. In another embodiment, instead of the two separate sensor rows with sensors distributed over the entire height of the seat at a distance of 1-5 cm, two membrane sensors with a width of from 4 to 10 cm are arranged over the entire seat height with a separation corresponding to the spinal column.

Furthermore, DE 10 2008 049 112 A1 discloses a capacitive textile electrode for measuring body functions and/or vital parameters of persons for vehicle applications, for example in a seat or a bunk, which electrode has a multilayer structure. This comprises two textile layers, each of which has an electrically conductive electrode region, a further textile layer being provided in order to establish a distance between the other two textile layers.

In general, the electrical conductivity of any clothing (or other material) between the skin of the person and the electrode plays an important role in the signal quality obtained during contactless electrocardiographic measurement. By way of example, when a person gets on/in a vehicle, some period of time may be required until the electrocardiographic sensor is able to record a reliable signal. This is due both to any electrostatic charge of the clothing and the low contact conductance thereof. The electrostatic charge may be discharged relatively slowly, as a result of which the electrostatic charge dominates and attenuates or covers the measurement signal. In general, the conductivity between the skin of the person to be examined and the electrode is substantially influenced by the moisture content of the clothing of the person situated therebetween. The moisture content of the clothing is in turn determined by the microclimate between the electrode surface and the skin of the person to be examined. Thus, for example, it may be the case in a dry surrounding climate, for example in a dry vehicle interior, that the clothing is likewise relatively dry. On the other hand, sweating by the person to be examined leads to a more moist or humid microclimate between the skin of the person and the electrode, leading to an improved signal quality.

### SUMMARY

The present disclosure is based on the object of specifying a sensor, a sensor array and a seat or a couch for a contactless electrocardiographic measurement of persons, preferably in the context of vehicle applications, by means of which reliable statements can be made about the bodily functions and/or vital parameters of the person, i.e. which are able to supply a reliable signal with high signal quality at all times.

It should be noted that the features specified individually in the claims may be combined with one another in any desired technologically meaningful way and disclose further embodiments of the invention. The description, in particular in conjunction with the Figures, characterizes and specifies the invention further.

According to the invention, a sensor for a contactless electrocardiographic measurement of a person comprises at least one electrically conductive, planar electrode, which comprises an outer or measurement surface facing the person and an opposite, inner surface facing away from the person and lying opposite to the outer surface. Within the meaning of the present invention, “contactless” should be understood to mean that the electrode does not come into direct contact with the skin of the person to be examined (the subject). By way of example, pieces of clothing may be arranged between the subject and the electrode. The electrode may also be electrically insulated from the subject by a layer of insulation lacquer.

Furthermore, provision is made for a moisture generator on the side of the inner surface of the electrode. Moreover, the electrode is permeable to moisture. In principle, any means or any device capable of releasing moisture under certain conditions, for example in the form of vapor or liquid droplets, may be used as a moisture generator. In this manner it is possible to automatically control the moisture content of the microclimate between the outer surface of the electrode and the skin of the subject, in particular in conjunction with a measurement and regulation apparatus. In particular, moisture which is able to penetrate the moisture-permeable electrode and thus increases the moisture content of the microclimate is released by the moisture generator in the case of a microclimate which is too dry. The moister microclimate improves the signal quality of the measurement signal recorded by the sensor since electrostatic charges can be discharged more quickly. Furthermore, a reliable measurement signal is obtained more quickly by the sensor according to the invention.

In accordance with an advantageous embodiment disclosed herein, the sensor moreover comprises at least one moisture sensor arranged on the inner surface of the electrode and a controller. The moisture sensor is connected to the controller and acquires the moisture content on the inner surface of the electrode. Furthermore, the controller is configured to control the moisture generator by means of an actuator, depending on the values acquired by the moisture sensor. Accordingly, the controller can cause the moisture generator to release more or less moisture. Thus, a desired moisture content of the microclimate between the outer surface of the electrode and the skin of the subject can be controlled or regulated in a targeted manner. Here, the control, regulation and measurement functions required for this are assumed by the controller.

A sensor array according to the invention comprises at least two sensors of the type described above. Within the meaning of the present invention, a sensor array should be understood to mean any type of arrangement of a plurality of said sensors.

In accordance with the present invention, a seat or a couch in a vehicle comprises at least one sensor array of the type according to the invention, as described above, for a contactless electrocardiographic measurement of a person situated on the seat or on the couch.

Further features and advantages of the invention emerge from the following description of exemplary embodiments of the invention which are not to be understood as being restrictive and which will be explained in greater detail in the following text, with reference being made to the drawing. In detail:

### BRIEF DESCRIPTION OF THE DRAWINGS

FIG. 1 schematically shows a sensor array and a seat for a vehicle according to the prior art,

FIG. 2 schematically shows a sensor according to the invention in accordance with a first embodiment,

FIG. 3 schematically shows a magnified view of the sensor of FIG. 2,

FIG. 4 schematically shows a sensor according to the invention in accordance with another embodiment,

FIG. 5 schematically shows a sensor according to the invention in accordance with a further embodiment,

FIG. 6 schematically shows a sensor according to the invention in accordance with a further embodiment,

FIG. 7 schematically shows a sensor according to the invention in accordance with a further embodiment,

FIG. 8 schematically shows a sensor according to the invention in accordance with a further embodiment,

FIG. 9 schematically shows a sensor according to the invention in accordance with a further embodiment,

FIG. 10 schematically shows a sensor according to the invention in accordance with a further embodiment,

FIG. 11 schematically shows a sensor according to the invention in accordance with a further embodiment,

FIG. 12 schematically shows a sensor according to the invention in accordance with a further embodiment, and

FIG. 13 schematically shows a sensor according to the invention in accordance with a further embodiment.

### DETAILED DESCRIPTION

As required, detailed embodiments of the present invention are disclosed herein; however, it is to be understood that the disclosed embodiments are merely exemplary of the invention that may be embodied in various and alternative forms. The Figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

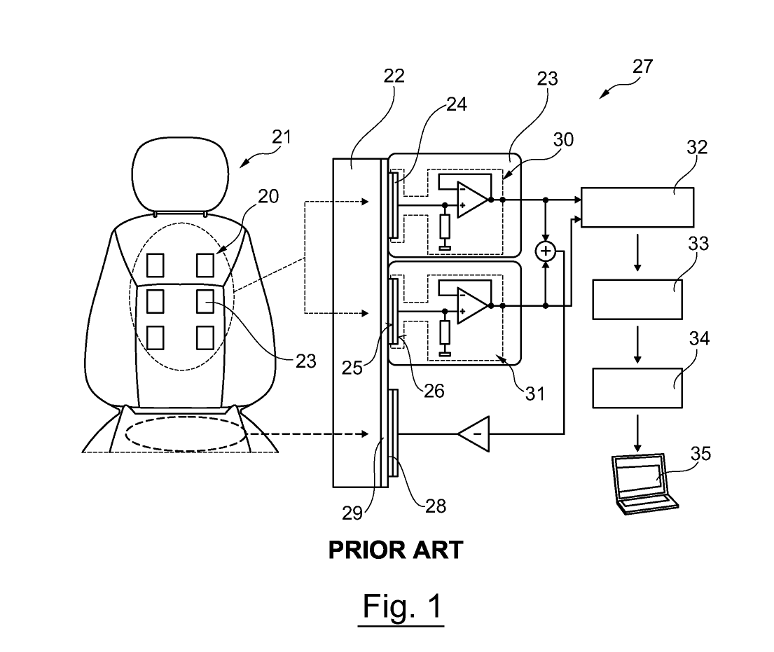

FIG. 1 schematically represents a sensor array 20 and a seat 21 for a vehicle for contactless electrocardiographic measurement on a person or subject 22, according to the prior art. As can be seen, the sensor array consists of a matrix arrangement of six sensors 23 arranged in a 3×2 matrix in a backrest of a vehicle seat, each of which sensors comprises a flat electrically conductive electrode 24. Another electrode, via which a reference potential is applied to the circuit, is furthermore arranged in the seat surface of the vehicle seat 21.

Each electrode 24 comprises an outer or measurement surface 25 facing toward the subject 22, and an inner or connection surface 26, facing away from the person and opposite the measurement surface 25, for the connection of a measuring device 27. As represented in FIG. 1, the measurement surface 25 of the individual electrodes 24 does not directly touch the skin of the subject 22. Rather, insulation 28 is applied on the measurement surface 25 of each electrode 24 in FIG. 1. Furthermore, the clothing 29 worn by the subject person also lies between the subject 22 and the measurement surface 25.

The measuring device 27 represented in FIG. 1 comprises one preamplifier 31, enclosed by shielding 30, per sensor 23. Furthermore, an instrument amplifier 32 amplifies the measurement signal registered by the electrodes 24 of the sensors 23, followed by a filtering and amplification unit 33 as well as an A/D converter 34. The digital measurement signal output by the A/D converter 34 may then be processed further in a suitable way, for example by means of a digital computer unit 35.

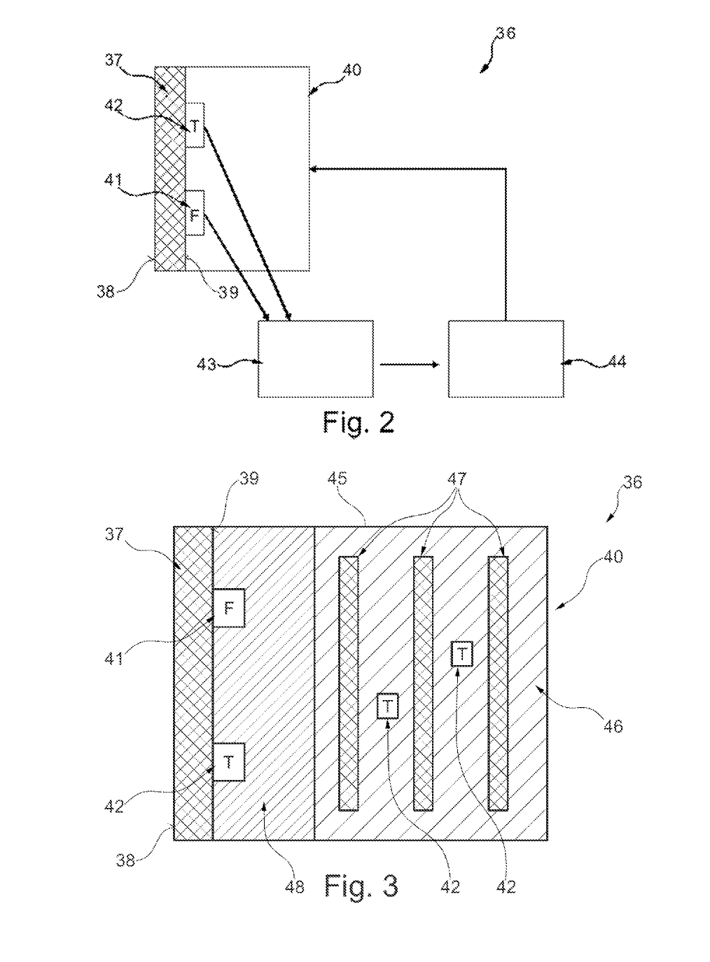

FIG. 2 schematically depicts a control loop for a sensor 36 according to a first embodiment of the invention. The sensor 36 comprises an electrically conductive, planar and moisture-permeable electrode 37, which comprises a measurement or outer surface 38 facing the person to be examined (the subject) and an inner surface 39 facing away from the subject and opposite from the outer surface 38. Moreover, the sensor 36 comprises a moisture generator 40 (not explicitly depicted in FIG. 2), which is provided on the side of the inner surface 39 of the sensor 36. Different embodiments of moisture generators 40, which are all able to release moisture under certain conditions, for example in the form of liquid vapor or liquid droplets, will still be described in more detail below in conjunction with the remaining Figures.

As is understood from FIG. 2, the control loop in the depicted exemplary embodiment comprises a moisture sensor 41 arranged on the electrode inner surface 39 and an (optional) temperature sensor 42 likewise arranged on the electrode inner surface 39. Both sensors 41 and 42 are connected to a controller 43, which controls the moisture generator 40 by means of an actuator 44. Accordingly, the controller 43 causes the moisture generator 40 to release more or less moisture, depending on the values determined by the sensors 41 and 42. Thus, a desired moisture content of the microclimate between the electrode outer surface 38 and the skin of the subject can be controlled or regulated in such a targeted manner that a reliable electrocardiographic measurement signal of the sensor 36 is obtained with good signal quality.

FIG. 3 depicts an embodiment of the sensor 36 from FIG. 2 in a magnified view. In particular, FIG. 3 depicts a possible embodiment of the moisture generator 40 in a detailed manner. The moisture generator 40 here comprises a chamber 45 containing a substance 46 which can store moisture and which can emit moisture when heated. By way of example, silica gel or a super absorbent polymer can be used as such a substance. A plurality of heating elements 47 arranged in the chamber 45 can be identified in FIG. 3. In the depicted embodiment, the heating elements 47 are completely surrounded by substance 46, and so said elements can heat the latter. Moreover, further temperature sensors 42, which can serve to avoid overheating within the chamber 45, are arranged between the heating elements 47. A spacer layer 48 which is able to transmit the moisture emitted by the moisture-storing substance may be inserted between the chamber 45 and the inner surface 39 of the electrode 37.

Although not depicted in FIG. 3, the moisture sensor 41 and the temperature sensors 42 are connected to the controller 43 as described in relation to FIG. 2. The controller controls the heating elements 47, which form the actuator 44 of the moisture generator 40 depicted in FIG. 2, in such a way that more or less moisture is released by the moisture generator 40, depending on the values established by the sensors 41 and 42. Thus, the moisture content of the microclimate between the outer surface 38 of the electrode 37 and the skin of the subject is controlled or regulated in such a targeted manner that a reliable electrocardiographic measurement signal with good signal quality is recorded via the outer surface 38 of the electrode 37.

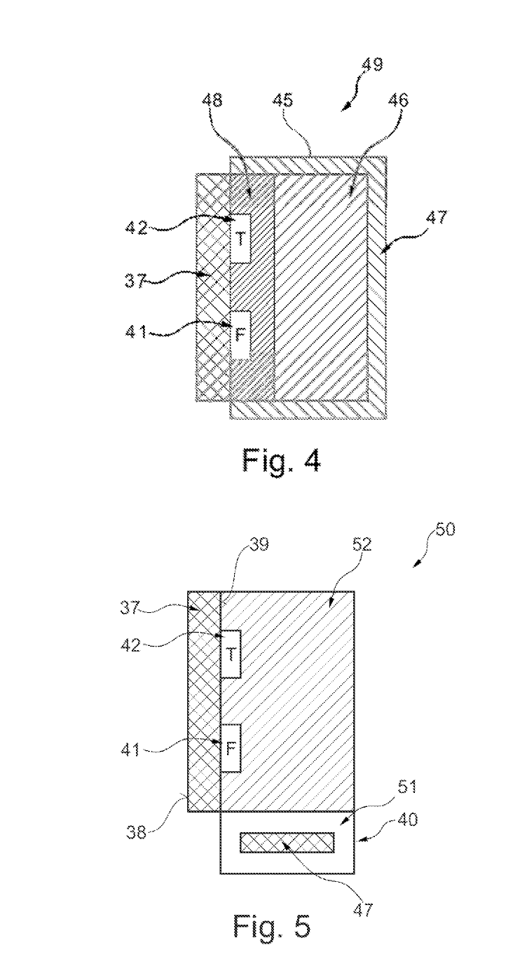

FIG. 4 depicts a sensor 49 according to the invention in accordance with another embodiment. The sensor 49 substantially differs from the sensor 36 depicted in FIG. 3 in terms of the arrangement of the heating elements 47. In the sensor 49 depicted in FIG. 4, the heating elements 47 are arranged on the rear walls and the side walls of the chamber 45, and so there is rear side and lateral heating of the chamber 45 filled with the substance 46 by means of the heating elements 47. It is likewise feasible to provide only the rear-side of the chamber 45 or only the side walls of the chamber 45 with heating elements 47. Further temperature sensors 42 can likewise be provided in the chamber 45 and/or on the heating elements 47, so as to avoid overheating of the heating elements 47 or of the chamber 45. In the same manner as described above in the explanation of FIG. 3, the sensor 49 can also be controlled or regulated by a controller 43, as depicted in FIG. 2, in conjunction with the sensors 41 and 42 and the actuator 44.

FIG. 5 depicts a further sensor 50 in accordance with a further embodiment in which the moisture generator 40 comprises a liquid reservoir 51 and at least one heating element 47 which heats the liquid reservoir 51. Vapor is generated in the liquid reservoir 51 with the aid of the heating element 47. The vapor passes through a cavity 52 provided between the inner surface 39 and the reservoir 51. Cavity 52 may contain a vapor-permeable material to conduct the vapor from the reservoir 51 to the inner surface 39 of the electrode 37.

Although this has not been depicted in FIG. 5, the moisture sensor 41 and the temperature sensor 42 are connected to the controller 43 as described in relation to FIG. 2. The controller controls the heating element 47, which forms the actuator 44 of the moisture generator 40 depicted in FIG. 2, in such a way that more or less moisture is released by the moisture generator 40, depending on the values established by the sensors 41 and 42. Thus, the moisture content of the microclimate between the outer surface 38 of the electrode 37 and the skin of the subject is controlled or regulated in such a targeted manner that a reliable electrocardiographic measurement signal with good signal quality is recorded via the outer surface 38 of the electrode 37.

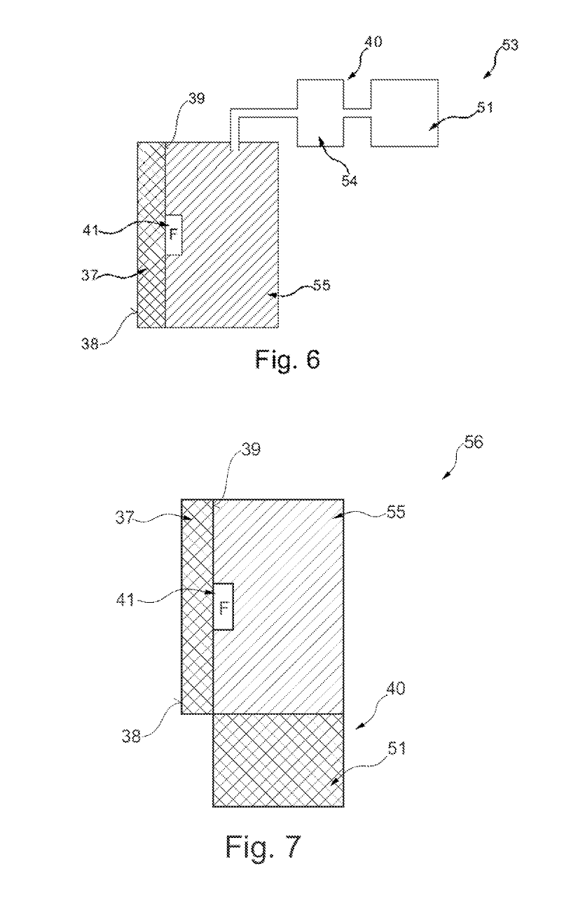

FIG. 6 depicts a further sensor 53 according to a further embodiment in which the moisture generator 40 comprises a liquid reservoir 51 and a pump 54 which pumps liquid from the reservoir 51. The water pumped from the reservoir 51 the pump 54 is conducted to the electrode inner surface 39 by a material 55 which can conduct or wick liquid, e.g. a sponge, such that said inner surface is moistened.

Although this has not been depicted in FIG. 6, the moisture sensor 41 is connected to the controller 43 described in relation to FIG. 2. The controller controls the pump 54, which forms the actuator 44 of the moisture generator 40 depicted in FIG. 2, in such a way that more or less moisture is released by the moisture generator 40, depending on the values established by the moisture sensor 41. Thus, the moisture content of the microclimate between the outer surface 38 of the electrode 37 and the skin of the subject is controlled or regulated in such a targeted manner that a reliable electrocardiographic measurement signal with good signal quality is recorded via the outer surface 38 of the electrode 37.

A sensor 56 according to a further embodiment is depicted in FIG. 7 wherein the material 55 which can conduct or wick liquid, preferably a sponge, is permanently dipped into the reservoir 51. The sponge 55 conducts the liquid from the reservoir 51 to the inner surface 39 of the electrode 37. In the exemplary embodiment depicted in FIG. 7, the moisture sensor 41 merely has a monitoring function. In this exemplary embodiment, there is no control or regulation of the release of moisture by the moisture generator.

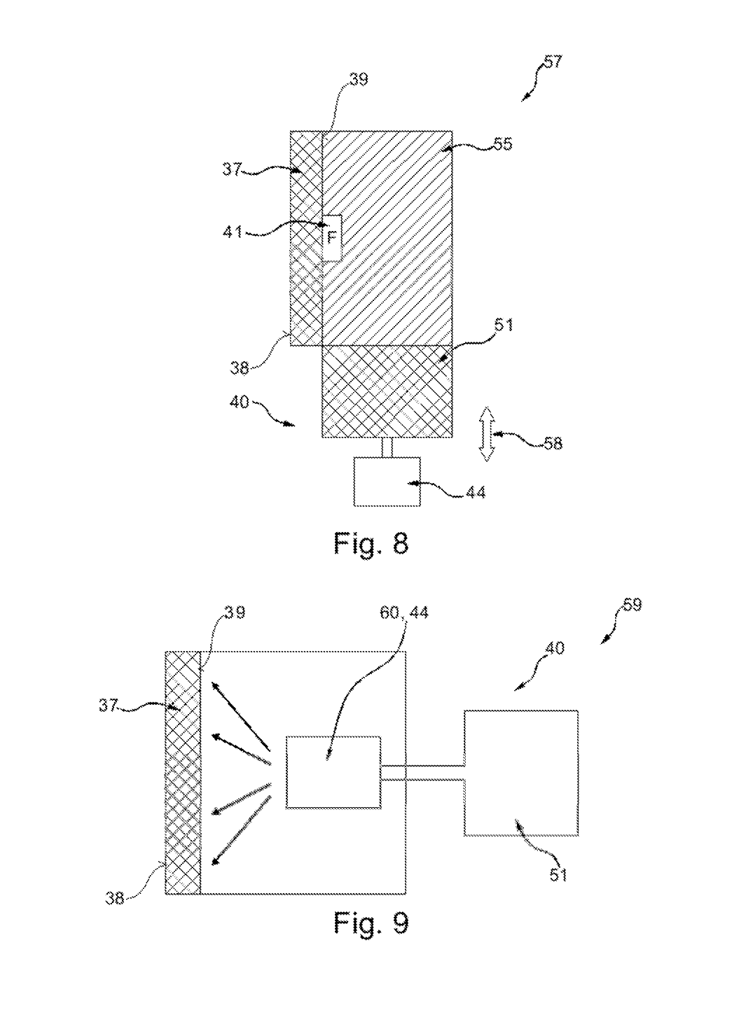

By contrast, such a control or regulation is made possible in the additional exemplary embodiment of a sensor 57 as depicted in FIG. 8. In this case, the actuator 44 serves to dip the material 55 which can conduct liquid, for example a sponge, to a greater or lesser extent into the liquid reservoir 51, depending on the degree of the desired moisture emission by the moisture generator 40. By way of example, the actuator 44 is able to move the sponge 55 or the liquid reservoir 51 along the movement trajectory 58 depicted in FIG. 8, and therefore able to determine the dipping-in depth of the sponge 55 in the liquid reservoir 51.

Although this has not been depicted in FIG. 8, the moisture sensor 41 is connected to the controller 43 described in relation to FIG. 2. The controller controls the actuator 44 of the moisture generator 40 in such a way that more or less moisture is released by the moisture generator 40, depending on the values established by the moisture sensor 41. Thus, the moisture content of the microclimate between the outer surface 38 of the electrode 37 and the skin of the subject is controlled or regulated in such a targeted manner that a reliable electrocardiographic measurement signal with good signal quality is recorded via the outer surface 38 of the electrode 37.

FIG. 9 depicts another embodiment of a sensor 59 in which the moisture generator 40 comprises a liquid reservoir 51 and at least one ultrasonic atomizer 60, which forms the actuator 44 of the moisture generator 40. The ultrasonic atomizer 60 atomizes the liquid stored in the liquid reservoir 51 and conducts the liquid mist to the inner surface 39 of the electrode 37 such that the latter is moistened as a result thereof. By controlling the ultrasonic atomizer 60 by means of the controller 43 (not depicted in FIG. 9) and by means of the sensors 41 and 42 (FIG. 2) (likewise not depicted here), it is possible to release more or less moisture by the moisture generator 40. Thus, the moisture content of the microclimate between the outer surface 38 of the electrode 37 and the skin of the subject is controlled or regulated in such a targeted manner that a reliable electrocardiographic measurement signal with good signal quality is recorded via the outer surface 38 of the electrode 37.

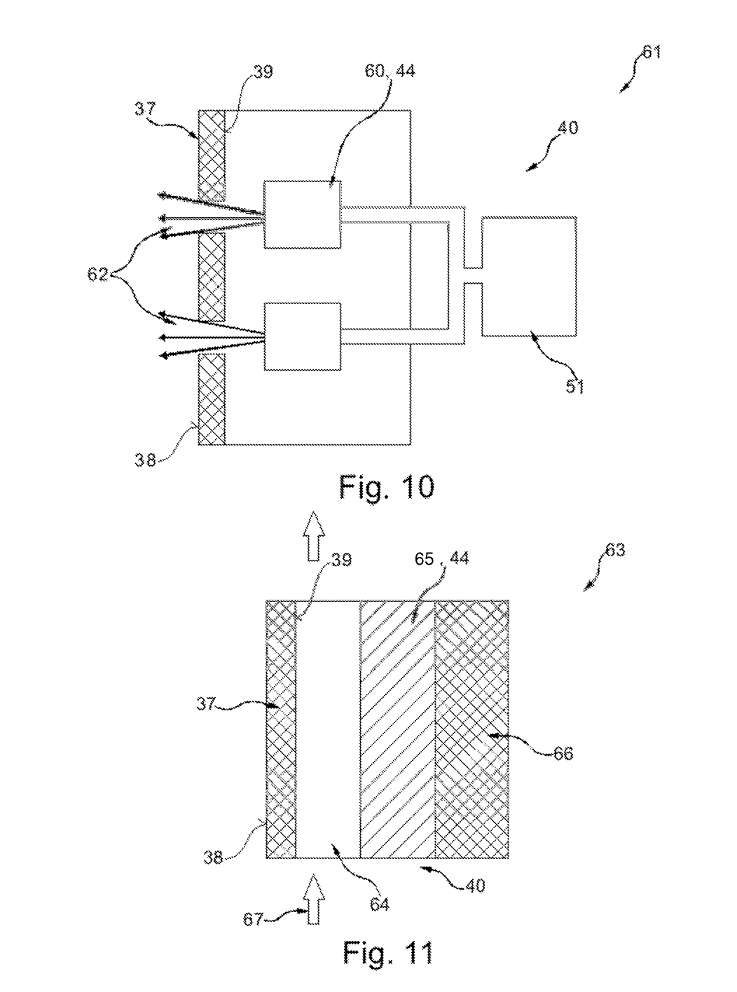

In the additional exemplary embodiment of a sensor 61 depicted in FIG. 10, the liquid mist generated by at least one ultrasonic atomizer 60 is not conducted to the inner surface 39 of the electrode 37 (as in FIG. 9), but rather directly in the direction of the subject by the sensor 61 or their clothing through openings 62 provided in the electrode 37. Here, the ultrasonic atomizer 60 is once again controlled as described above by means of the controller 43 (FIG. 2) (not depicted in FIG. 10).

Instead of the ultrasonic atomizer 60 used in the sensors 59 and 61, use can for example likewise be made of a pump and a spray nozzle as actuators 44 of the moisture generator 40.

FIG. 11 depicts a further embodiment of a sensor 63 in which the moisture generator 40 comprises at least one Peltier element 65 and a material 64 which is both permeable to air and able to store moisture. The air-permeable moisture-storing material 64 is arranged adjacent to the electrode inner surface 37. The Peltier element 65 is arranged adjacent to the air-permeable moisture-storing material 64. In the exemplary embodiment depicted in FIG. 11, a cooling body 66 is moreover arranged adjacent to the Peltier element 65. The cooling body 66 serves to supply heat to, or dissipate heat from, the Peltier element 65.

The inner surface 39 of the electrode 37 is moistened by alternately a) cooling the air-permeable moisture-storing material 64, whereby water is obtained by condensation from an air flow 67 passing through the cooled material 64, and b) heating the material 64 to release the condensed water stored in the material 64. The heating and cooling is brought about by the Peltier element 65.

Here, the material which can store moisture can also be separated laterally from the surroundings; in this case, the regeneration is brought about by moisture or a moisture-containing air flow passing through the electrode permeable to moisture.

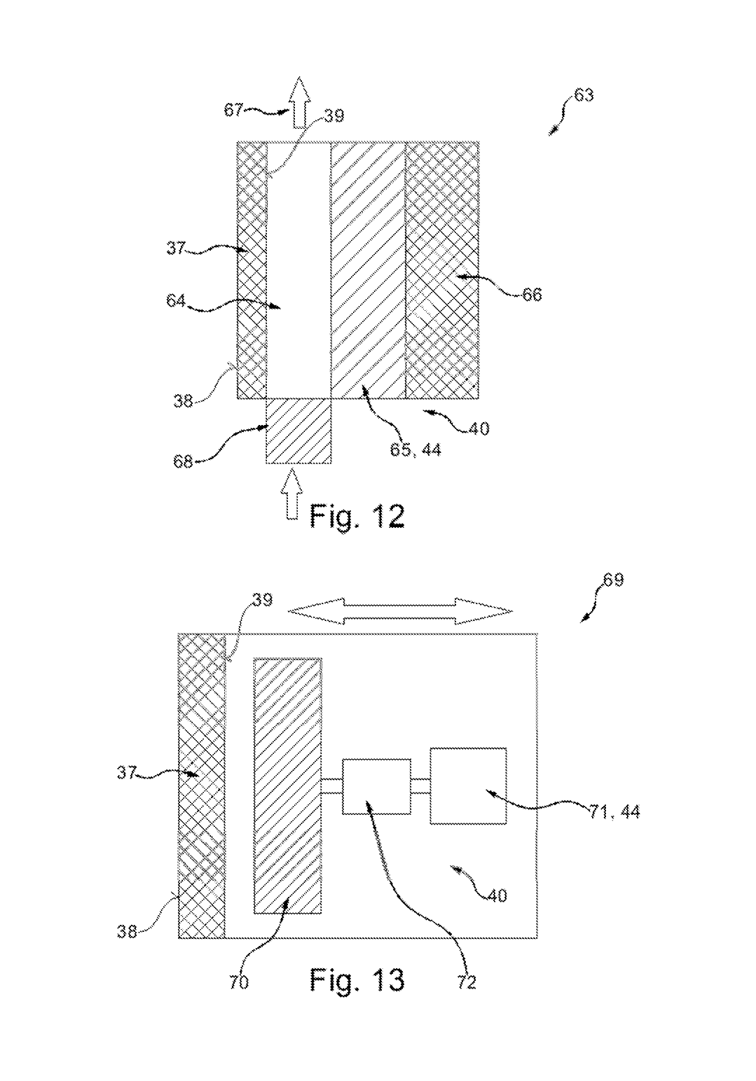

The process of obtaining water at the air-permeable moisture-storing material 64 can additionally be supported by a ventilator 68, as is depicted in the exemplary embodiment of the sensor 63 as shown in FIG. 12. Here, the application of surrounding air to the material 64 is performed by the ventilator 68 and preferably is only carried out when obtaining water, i.e. in the cooling phase of the Peltier element 65. When releasing the water stored in the material 64 by heating by means of the Peltier element 65, the ventilator 68 is not operating so there is no application of surrounding air.

The Peltier element 65 is once again controlled by means of the controller 43 (FIG. 2) (not depicted in FIG. 12), as a result of which more or less moisture can be released by the moisture generator 40. Thus, the moisture content of the microclimate between the outer surface 38 of the electrode 37 and the skin of the subject is controlled or regulated in such a targeted manner that a reliable electrocardiographic measurement signal with good signal quality is recorded via the outer surface 38 of the electrode 37.

FIG. 13 depicts a further exemplary embodiment of a sensor 69 in which the moisture generator 40 comprises a compressible material 70 which can store water, for example a sponge, and a displacement apparatus 71, which is e.g. motor driven, for pressing the compressible water-storage material 70 against the inner surface 39 of the electrode 37, which is moistened to a greater or lesser extent depending on the contact pressure applied against the inner surface 39. By way of example, the contact pressure can be measured by a force sensor 72 arranged between the compressible water-storage material 70 and the displacement apparatus 71. The force sensor 72 is expediently connected to the controller 43 (FIG. 2) (not depicted in FIG. 13), which in turn controls the displacement apparatus 71 of the moisture generator 40 in such a way that more or less moisture can be released by the moisture generator 40 depending on the values established by the force sensor 72. Thus, the moisture content of the microclimate between the outer surface 38 of the electrode 37 and the skin of the subject is controlled or regulated in such a targeted manner that a reliable electrocardiographic measurement signal with good signal quality is recorded via the outer surface 38 of the electrode 37.

Once again, various options are feasible for moistening the water-storage material 70, for example the already described option by means of a pump and a water reservoir.

The sensor according to the invention, the sensor array and the seat or the couch were explained in more detail on the basis of several exemplary embodiments depicted in the Figures. However, the sensor, the sensor array and the seat or couch are not restricted to the embodiments described herein, but rather also comprise further embodiments with the same effect.

In a preferred embodiment, the sensor according to the invention, the sensor array and the seat or the couch are used in a vehicle, in particular in a motor vehicle, for a contactless electrocardiographic measurement of a person.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A contactless electrocardiographic sensor, comprising:
an electrode formed of a moisture-permeable material and having a measurement surface and an opposite surface;
a moisture generator supplying moisture to the opposite surface; and
a controller activating the moisture generator to increase a moisture content of a microclimate at the measurement surface.

2. The sensor of claim 1, further comprising:
a moisture sensor for detecting the moisture content, the controller activating the moisture generator based at least is part on signals received from the moisture sensor.

3. The sensor of claim 2, further comprising a temperature sensor, the controller activating the moisture generator based at least is part on signals received from the temperature sensor.

4. The sensor of claim 1, wherein the moisture generator comprises a heating element heating a source of moisture when activated by the controller.

5. The sensor of claim 4, wherein the source of moisture is a moisture-storing substance which emits moisture when heated.

6. The sensor of claim 1, wherein the moisture generator comprises a pump activated by the controller to pump liquid from a reservoir to the electrode.

7. The sensor of claim 1, wherein the moisture generator comprises an actuator activated by the controller to vary an amount of a liquid-conducting material in contact with liquid contained in a reservoir.

8. The sensor of claim 1, wherein the moisture generator comprises an ultrasonic atomizer for atomizing liquid contained in a reservoir.

9. The sensor of claim 1, wherein the moisture generator comprises a Peltier element operable to warm and thereby release moisture from a moisture-storing material adjacent to the electrode.

10. A contactless electrocardiographic sensor, comprising:
an electrode formed of a moisture-permeable material and having a measurement surface and an opposite surface;
a moisture generator supplying moisture to the opposite surface;
a moisture sensor for detecting a moisture content of a microclimate at the measurement surface; and
a controller receiving signals from the moisture sensor and activating the moisture generator based upon the signals to control the moisture content.

11. A seat for a motor vehicle having a sensor array for contactless electrocardiographic measurement, the sensor array comprising:
an electrode mounted to the seat, formed of a moisture-permeable material, and having a measurement surface for registering a measurement signal and an opposite surface;
a moisture generator supplying moisture to the opposite surface; and
a controller activating the moisture generator to increase a moisture content of a microclimate at the measurement surface.

12. The seat of claim 11, further comprising:
a moisture sensor for detecting the moisture content, the controller activating the moisture generator based at least is part on signals received from the moisture sensor.

13. The seat of claim 11, further comprising a temperature sensor, the controller activating the moisture generator based at least is part on signals received from the temperature sensor.

14. The seat of claim 11, wherein the moisture generator comprises a heating element heating a source of moisture when activated by the controller.

15. The seat of claim 14, wherein the source of moisture is a moisture-storing substance which emits moisture when heated.

16. The seat of claim 11, wherein the moisture generator comprises a pump activated by the controller to pump liquid from a reservoir to the electrode.

17. The seat of claim 11, wherein the moisture generator comprises an actuator activated by the controller to vary an amount of a liquid-conducting material in contact with liquid contained in a reservoir.

18. The seat of claim 11, wherein the moisture generator comprises an ultrasonic atomizer for atomizing liquid contained in a reservoir.

19. The seat of claim 11, wherein the moisture generator comprises a Peltier element operable to warm and thereby release moisture from a moisture-storing material adjacent to the electrode.

20. The seat of claim 11, wherein the moisture generator comprises a device for pressing a compressible material against the opposite surface of the electrode to release liquid stored in the compressible material.
